(19) United States
(12) Patent Application Publication
(10) Pub. No.: US 2025/0032367 A1
(43) Pub. Date: Jan. 30, 2025

(54) BARIUM SILICATE CEMENT FOR DENTAL AND MEDICAL APPLICATIONS, AND METHODS OF USE

(71) Applicants: Quanzu Yang, Vancouver (CA); Yuhang George Yang, Burnaby (CA)

(72) Inventors: Quanzu Yang, Vancouver (CA); Yuhang George Yang, Burnaby (CA)

(21) Appl. No.: 18/225,570

(22) Filed: Jul. 24, 2023

(51) Int. Cl. A61K 6/853; A61K 6/864

(52) U.S. Cl. CPC A61K 6/853; A61K 6/864

(57) ABSTRACT

A cement is provided for use in medical and dental procedures. The cement includes a barium silicate compound, and is formulated to be biocompatible and bioactive. The barium silicate compound is in the form of a powder, and includes one or more of a di-barium silicate, a tri-barium silicate, a mono-barium aluminate, a di-barium aluminate, and a tri-barium aluminate. The cement can optionally be provided as a premixed paste in which the cement powder is mixed with a nonaqueous liquid.

### BACKGROUND

### Technical Field

This disclosure relates generally to cement formulations and methods for use in medical and dental applications, and more particularly to barium-based cements.

### Description of the Related Art

MESTIERI et al. did research on Biocompatibility and bioactivity of calcium silicate-based endodontic sealers in human dental pulp cells (J Appl Oral Sci. 2015 Sep.-Oct; 23(5): 467-471). The research results indicated that calcium silicate materials are biocompatible and bioactive. Also, Huang et al. did research on Substitutions of strontium in bioactive calcium silicate bone cements to stimulate osteogenic differentiation in human mesenchymal stem cells (J Mater Sci Mater Med. 2019 Jun. 4; 30(6):68.). Applicant is not aware of any studies or reports relating to the biological properties of Barium silicates.

Torabinejad et al. (U.S. Pat. No. 5,415,547) discloses tooth filling material and method of use. An improved method for filing and sealing tooth cavities involves the use of a cement composition which exhibits several advantages over existing orthograde and retrograde filling materials, including the ability to set in an aqueous environment. In a preferred embodiment, the cement composition comprises Portland cement, or variations in the composition of such cement, which exhibit favorable physical attributes sufficient to form an effective seal against reentrance of infectious organisms.

Yang et al. (U.S. Pat. No. 8,475,811) discloses a premixed biological hydraulic cement paste composition. The premixed cement paste remains fluid while stored in a hermetically sealed condition, but hydrates and hardens when placed in a physiological environment. The cement paste includes at least one calcium silicate compound and at least one substantially water-free liquid carrier mixed with the calcium silicate compound; the substantially water-free liquid carrier prevents hydration of the mixture during storage. Calcium silicate compounds hydrate to produce calcium silicate hydrogel and calcium oxide. The calcium silicate hydrogel is the primary structure of the cement.

Chow et al. (U.S. Pat. No. 9,101,436) discloses endodontic filling materials and methods. A method for filling a dental root canal includes providing a hydrosetting filling material and inserting the hydrosetting filling material into the dental root canal so that the material sets in the root canal to form a biocompatible filling. The hydrosetting filling material comprises a hydrogel former and a filler. The hydrogel former is at least one of a reactive organic hydrogel former, an inorganic hydrogel former, or a non-reactive organic hydrogel former. Plural filling material precursor compositions that collectively contain hydrogel formers and fillers may be provided. Chow teaches the use of calcium silicates and sodium silicates as the inorganic hydrogel formers.

Jin (U.S. Pat. No. 11,110,037) discloses the composition with nanogels of highly-branched discrete polymeric particles and fillers. Jin describes a tremendous need to reduce polymerization shrinkage and the disrupting stress of dental composites in order to create advanced composite restorations. Barium silicate is mentioned as a filler for a polymer composition.

Jia et al. (U.S. Pat. No. 7,303,817) discloses a dental filling material comprising an inner core and outer layer of material disposed and surrounding the inner core, both the inner core and outer layer of material each containing a thermoplastic polymer. The thermoplastic polymer may be biodegradable, and a bioactive substance may also be included in the filling material. Jia teaches a barium silicate filler material.

Wagh et al. (U.S. Pat. No. 7,083,672) discloses a method and product for a phosphosilicate slurry for use in dentistry and related bone cements. The composition is produced by combining a mixture of a substantially dry powder component with a liquid component. The substantially dry powder component comprises a sparsely soluble oxide powder, an alkali metal phosphate powder, a sparsely soluble silicate powder, with the balance of the substantially dry powder component comprising at least one powder selected from the group consisting of bioactive powders, biocompatible powders, fluorescent powders, fluoride releasing powders, and radiopaque powders. The liquid component comprises a pH modifying agent, a monovalent alkali metal phosphate in aqueous solution, the balance of the liquid component being water. Mono barium silicate is mentioned as a sparsely soluble (in water) powder. Wagh also refers to a barium silicate glass as a component of filler materials.

### BRIEF SUMMARY

A cement is provided for use in medical and dental procedures. The cement includes a barium silicate compound, and is formulated to be biocompatible and bioactive. The barium silicate compound is in the form of a powder, and includes one or more of a di-barium silicate, a tri-barium silicate, a mono-barium aluminate, a di-barium aluminate, and a tri-barium aluminate. The cement can optionally be provided as a premixed paste in which the cement powder is mixed with a nonaqueous liquid carrier.

According to an embodiment, a material for use in medical applications is provided, which includes a premixed paste having a primary cement compound that includes one or more barium cements that are capable of a hydration reaction with water to form a hydrogel, and a nonaqueous liquid carrier that is hydrophilic and able to undergo an exchange with a water-based liquid when the premixed paste is exposed to an environment where water-based liquids are present.

According to an embodiment, the premixed paste is packaged in a hermetically-sealed container.

According to an embodiment, the one or more barium cements includes one or more of di-barium silicate (Ba₂SiO₄), tri-barium silicate (Ba₃SiO₅), mono-barium aluminate, di-barium aluminate, and tri-barium aluminate.

According to another embodiment, the primary cement compound comprises at least 2% of the barium cement, by weight.

According to an embodiment, a method of using a cement material in medical applications is provided, including preparing a cement, which includes a a barium cement powder, for use in a dental procedure, performing the dental procedure, including introducing the cement into a patient during the performance of the procedure, and reacting the cement with an aqueous solution to form a hydrogel.

According to an embodiment, the preparing a cement, which includes a barium cement powder, for use in a dental procedure comprises preparing the cement, which includes a barium cement powder comprising one or more of a di-barium silicate (Ba₂SiO₄), a tri-barium silicate (Ba₃SiO₅), a mono-barium aluminate, a di-barium aluminate, and a tri-barium aluminate.

According to another embodiment, the reacting the cement with an aqueous solution comprises binging the cement into contact with the aqueous solution.

According to an embodiment the bringing the cement into contact with the aqueous solution comprises mixing the cement with the aqueous solution prior to introducing the cement into the patient.

According to an embodiment the binging the cement into contact with the aqueous solution comprises binging the cement into contact with biological fluids present within the patient.

According to an embodiment, the preparing a cement, which includes a barium cement powder, for use in a dental procedure comprises removing from a package a premixed paste that includes the barium cement powder mixed with a nonaqueous liquid.

According to a further embodiment, the introducing the cement into a patient during the performance of the procedure comprises introducing the premixed paste into the patient.

According to an embodiment, the reacting the cement with an aqueous solution comprises, after introducing the premixed paste into the patient, exchanging biological fluids of the patient with the nonaqueous fluid of the premixed paste.

### BRIEF DESCRIPTION OF THE DRAWINGS

FIG. 1A is a scanning electron microscope (SEM) micrograph image showing a surface plan view of a barium silicate cement.

FIG. 1B is a SEM micrograph image showing the surface morphology of a sample of barium silicate cement hydrogel after immersion in simulated body fluid (SBF) for 7 days.

FIG. 1C is a SEM micrograph image showing a cross-sectional view of the barium silicate sample of FIG. 1B, showing the thickness of a layer of hydroxyapatite that formed on the barium silicate sample during the immersion.

FIGS. 2A-2C are substantially similar to FIGS. 1A-1C, respectively, except that they show images of a tricalcium cement hydrogel control sample, in corresponding views.

### DETAILED DESCRIPTION

In the following detailed description, reference is made to the accompanying drawings, which form a part hereof. Other embodiments may be used and/or other changes may be made without departing from the spirit or scope of the disclosure.

Hereafter, where relative quantities of components, ingredients, etc., are given in percentages, it will be by weight, unless otherwise indicated. As used herein, the term biocompatible is used to refer to materials that are well-tolerated by the biological systems they come into contact with. The term bioactive is used to refer to materials that produce a local physiological response in a system in which it is used, typically through physical or chemical action.

According to an embodiment, a barium cement is provided, which is biocompatible, bioactive, and has high radiopacity, and which is suitable for dental and medical applications. According to a further embodiment, the barium cement compound hydrates with water and/or moisture, to produce a barium hydrogel compound, as a structural component, and barium hydroxide. The barium cement comprises a primary compound that includes hydratable barium silicates and/or barium aluminates. The primary cement compound includes one or more of barium silicate (BaₓSiOₓ₊₂ where x>1), di-barium silicate (Ba₂SiO₄), tri-barium silicate (Ba₃SiO₅), mono-barium aluminate, di-barium aluminate, and tri-barium aluminate.

The term hydratable refers to compounds that are capable of reacting with water to form hydrogels. It should be noted that some compounds are not typically hydratable. For example, mono-barium silicate (BaSiO₃) is soluble in water, so that instead of reacting to form a hydrogel, it dissolves. However, in some embodiments mono-barium silicate may comprise a portion of a barium cement compound when in combination with other, hydratable compounds.

According to an embodiment, the primary cement compound comprises at least 2% of the total composition of the barium cement. According to another embodiment, the primary cement compound comprises at least 10% of the total composition of the barium cement. According to a further embodiment, the primary compound comprises at least 20% of the total composition of the barium cement, while in another embodiment, the primary compound comprises at least 40% of the total composition of the barium cement, and in a further embodiment the primary compound comprises at least 60% of the total composition of the barium cement.

According to an embodiment, a secondary cement compound is incorporated into the barium cement to improve physical, chemical, and/or biological properties of the barium cement, as described in more detail below.

The primary and secondary cement compounds may be provided in the form of fine powder which, in a water or water-free medium can be formed into a paste that is convenient for use in many applications.

According to an embodiment, when cement compounds of the barium cement are hydrated by contact with water, they form a hydrogel as a structural element, which, as in the embodiment discussed below, may be mixed with other materials, particularly particulates, that are bound in a matrix formed when the cement cures. This is in contrast with some prior art filler compounds that employ various cements that act as fillers. The inventors understand the term filler as referring to a material that is present as discrete particles that provide strength and/or mass, but that are suspended in a matrix of another material, as an aggregate. In some embodiments, the hydrogel acts as a matrix that encapsulates or suspends particles of addition elements, such as other secondary compounds and/or phases.

According to research conducted or directed by the inventors, a barium compound hydrogel, such as those described herein with reference to various embodiments, and as explained in more detail below in the description of Example 2, are biocompatible and bioactive. In particular, in the presence of body fluids, barium compound hydrogels form a layer of hydroxyapatite, which can act as an anchor for and encourage the formation of new bone growth in appropriate circumstances (in many prior art formulations, hydroxyapatite is include as a separate ingredient, for similar purposes). This characteristic of barium compound hydrogels is of particular value in dental and medical applications where a patient has experienced bone damage or loss. Another advantage of barium cement is its high radiopacity, which is a very important property for dental restorations, root canal filling, microsurgery, pulp capping, dentin repairing applications, etc. It not only helps diagnose defects of treatment such as fractures, voids, over contouring, missing proximal contact, marginal imperfections, secondary caries and more, but it is also helpful in documenting treatment for clinical follow-up and continuous treatment.

As previously noted, in some embodiments the barium cement includes one or more secondary cement compounds. These secondary cement compounds may include calcium silicate compounds, calcium aluminate compounds, barium aluminate compounds, calcium phosphate compounds, strontium silicate compounds, strontium aluminate compounds, alkali silicate compounds, alkali aluminate compounds, magnesium silicate compounds, strontium aluminate compounds, lithium silicate compounds, sodium silicates compounds, potassium silicate compounds, ruthenium silicate compounds, etc. For example, according to an embodiment, tricalcium silicate powder is incorporated into the barium cement. When the cement powders are mixed with water, tricalcium silicate and barium silicate will simultaneously react with water to produce a calcium silicate/barium silicate hydrogel and calcium/barium hydroxide.

According to an embodiment, a barium cement is provided that includes one or more secondary cement compounds in the range of 2%-80% of the total composition. According to another embodiment, the one or more secondary cement compounds comprise less than 50% of the total cement composition.

Another advantage of cement materials that include barium silicates is a high mechanical strength.

Referring in further detail to the secondary cement compounds, the calcium silicate compounds may include calcium silicate (CaSiO₃), dicalcium silicate (Ca2SiO₄), and/or tricalcium silicate (CasSiO₃); the barium aluminate compound may include mono-barium aluminate (BaO.Al₂O₃), di-barium aluminate (2BaO.Al₂O₃), and/or tri-barium aluminate (3BaO.Al₂O₃); the calcium phosphate compounds may include mono-calcium phosphate, dicalcium phosphate, tricalcium phosphate, octacalcium phosphate, amorphous calcium phosphate, and/or hydroxyapatite; the strontium silicate compounds may include mono-strontium silicate (SrSiO₃), di-strontium silicate (Sr₂SiO₄), and/or tri-strontium silicate (Sr₃SiO₅); the alkali silicate compounds may include alkali silicates, alkali disilicates, and/or alkali trisilicates; the magnesium silicate compounds may include mono-magnesium silicate (MgSiO₃), di-magnesium silicate (Mg₂SiO₄), and/or tri-magnesium silicate (Mg₃SiO₅); the lithium silicates compounds may include lithium silicate, lithium disilicate, and/or lithium trisilicate; the sodium silicates compounds may include sodium silicate, sodium disilicate, and/or sodium trisilicate; the potassium silicate compounds may include potassium silicate, potassium disilicate, and/or potassium silicate; and the ruthenium silicate compounds may include ruthenium silicate, ruthenium disilicate, and/or ruthenium trisilicate. The compounds listed above are not intended as a complete or comprehensive list but instead as representative examples. A person having ordinary skill in the art will recognize other cement compounds that may also be incorporated without departing from the scope of this disclosure. According to further embodiments, additional secondary compounds and/or secondary phases are incorporated into the barium cement to improve physical, chemical, and/or biological properties of the cement. These may include, for example, ceramics, ceramic fibers, polymers and polymer fibers, metals, metal salts, metal oxides, hydroxide compounds, non-oxide ceramics, biopolymers, and mixtures thereof. According to an embodiment, secondary compounds and secondary phases comprise less than 80% of the total composition of the barium cement.

The metal salts may include, calcium salts, sodium salts, iron salts, magnesium salts, barium salts, strontium salts, potassium salts, zinc salts, phosphates, carbonate, sulfates, silicates, aluminates, and/or hydrogen salts; the metal oxides may include calcium oxides, sodium oxides, iron oxides, magnesium oxides, barium oxides, strontium oxides, potassium oxides, zinc oxides, zirconium oxide, titanium oxide, tantalum oxides, aluminum oxide, tungsten oxide, bismuth oxide, nickel oxides, cobalt oxides, hafnium oxides, yttrium oxides, silver oxide, and/or gold oxides; the metals may include stainless steel, iron, titanium, tantalum, aluminum, tungsten, bismuth, nickel, cobalt, hafnium, yttrium, silver, gold, platinum, and/or alloys thereof; the non-oxides may include silicon carbide, silicon nitride, borate silicon, titanium nitride, titanium nitride, nitride-oxide and/or titanium; the biopolymers may include biodegradable biopolymers and/or non-biodegradable polymers. Furthermore, according to an embodiment, the barium cement may comprise gutta percha powder for improving sealing abilities and re-treatment abilities in dental applications.

For some dental and orthopedic applications, radiopaque materials may be added to the barium cement composition to improve absorption of X-rays and thus visibility of the implant in X-ray images. The radiopaque materials that may be used include, for example, metals, metal oxides, salts, non-oxides, and mixtures thereof. Examples of such additive materials include barium sulfate, zirconium oxide, bismuth oxide, tantalum oxide, tantalum, titanium, stainless steel, alloys, and mixtures thereof, which, according to an embodiment, make up less than about 70% of the cement powder composition.

According to another embodiment, admixtures are included in the barium cement before or during mixing to provide or improve selected properties of the cement, such as to reduce water, to modify the properties of the hardened cement; to ensure the quality of the barium cement during mixing, transporting, placing, and/or curing; and to overcome certain emergencies during barium cement operations. These admixtures can be organized into four classes: water-reducing, retarding, accelerating, and plasticizers (superplasticizers).

Water-reducing admixtures usually reduce the required water content for a barium cement mixture by about 5 to 10 percent. Consequently, barium cement containing a water-reducing admixture needs less water to reach a required flowability than untreated cement. The treated cement can have a lower water-cement ratio. This also enables the production of a higher strength barium cement can without increasing the amount of cement.

Retarding admixtures, which slow the setting rate of barium cement, are used to counteract the accelerating effect of hot weather on concrete setting. High temperatures often cause an increased rate of hardening which makes placing and finishing difficult. Retarders keep the barium cement workable during placement and delay the initial set of barium cement. Most retarders also function as water reducers and may entrain some air in barium cement.

Accelerating admixtures increase the rate of early strength development, reduce the time required for proper curing and protection, and speed up the start of finishing operations. Accelerating admixtures are especially useful for modifying the properties of barium cement in cold weather.

Superplasticizers, also known as plasticizers or high-range water reducers (HRWR), can reduce water content by 12 to 30 percent and can be added to barium cement with a low-to-normal flowability and water-cement ratio to make high-slump flowing cement. Flowing barium cement is a highly fluid but workable cement that can be placed with little or no vibration or compaction. The effect of superplasticizers lasts only 30 to 60 minutes, depending on the formulation and dosage rate, and is followed by a rapid loss in workability. As a result of the flowability loss, superplasticizers are usually added to barium cement just prior to clinical applications, rather than as part of a premixed paste.

According to an embodiment, chemical admixtures include lignosulfonate (SLS), calcium Lignosulfonate (CLS), sodium naphthalene sulfonate (SNF), polycarboxylate superplasticizer, etc. According to an embodiment, these chemical admixtures comprise less than 10% of the total cement composition.

According to an embodiment, a method is provided for treating, filling and sealing cavities in teeth in which the filling material satisfies the existing need by providing an improved seal against invading bacteria. The present method can be used for both human and veterinarian applications.

Embodiments of the invention provide many advantages over prior dental filling and sealing materials. In an embodiment, barium cement sets in the presence of moisture and blood, and is therefore easily applied and suitable for use in moist environments such as the mouth. This is particularly beneficial when used as a retrograde filling material where fluid and blood are often difficult to control. Additionally, barium cement has a high radiopacity. It is not typically necessary to add other radiopaque materials to the composition. The barium cement composition of the disclosed embodiments is also compatible with surrounding biological tissue. This is particularly beneficial when it is in direct contact with periapical tissue, such as, for example, when used as a retrograde filling material.

According to an embodiment, a barium cement is provided that is formulated primarily for use in dental applications. Selection of the particular formulation, including primary cement compounds, secondary cement compounds, other secondary compounds and/or secondary phases, admixtures, etc., are case specific, i.e., design choices based on a desired combination of characteristics of the cement. Such selection is within the abilities of a person having ordinary skill in the art. According to another embodiment, a barium cement is is provided that is formulated primarily for use in non-dental medical applications, and the specific formulation is similarly subject to design choices driven by the particular characteristics desired for the specific application.

According to another embodiment, a premixed barium cement paste is provided, for improving the clinical handling ability of materials. The premixed barium cement paste comprises at least one hydratable barium cement compound substantially as described with reference to any of the previous embodiments, and at least one substantially water-free non-aqueous liquid carrier. The premixed barium cement paste is configured to be placed in a biological environment, whereupon the non-aqueous liquid of the composition undergoes an exchange with water from the biological environment, which reacts with the hydratable barium cement compounds to produce a barium compound hydrogel and barium hydroxide.

According to an embodiment, the hydratable barium compounds are in the range of 2%-90% of the premixed paste, while the non-aqueous liquid of the present invention is the range of 5%-50% of premixed paste. According to an embodiment, the non-aqueous liquid carrier is a hydrophilic liquid, to facilitate the exchange with water in the biological environment, by a diffusing process. The non-aqueous liquids may include one or more of ethyl alcohol, ethylene glycol, polyethylene glycol (PEG), glycerol liquid, glycerin, liquid organic acids, water-soluble vegetable oil, water-soluble animal oil, fish oil, water-soluble silicon oil, dimethyl sulfoxide, propylene glycol, etc.

According to an embodiment, the premixed paste is packaged in a hermetically sealed container. The container prevent prevents the paste from contacting water, either in liquid form or, particularly, as airborne vapor, which might otherwise gradually degrade the condition of the cement in the paste. The hermetic packaging enables the premixed paste to remain unused for extended periods without undergoing significant deterioration over time. Accordingly, the packaged paste is fully compatible with modern commercial distribution systems, able to be warehoused and transported by manufacturers, distributors, and end users without requiring special treatment, handling, or other considerations that might otherwise increase the inconvenience and/or cost to an end user.

According to a further embodiment, the premixed paste is packaged in single-dose quantities, i.e., in quantities that are likely to be sufficient for most situations. Single-use packaging can provide a number of advantages, particularly for an end user. For example, it can reduce the likelihood of contamination, such as can occur when the paste from one package is used to treat multiple patients; it can reduce spoilage of paste that only a portion of the paste in a package is used, so that the remainder must be either returned to storage or discarded.

As used herein the term substantially water-free means waterless or containing water in an amount sufficiently small that, in a premixed paste with a barium cement compound, the barium cement compounds doesn't result in the significantly change of the paste consistency and/or setting time when kept in a hermitically-sealed condition.

According to an embodiment, one or more additional secondary compounds are incorporated into the premixed cement paste. The additional secondary compounds may include tricalcium aluminate (3CaO.Al₂O₃); tetracalcium aluminoferrite (4CaO.Al₂O₃.Fe₂O₃); calcium oxide; ferrite oxide; calcium sulfate dihydrate (CaSO₄.2H₂O); sodium salts; magnesium salts; and/or strontium salts, and comprise less than 30% by weight of the cement in the premixed paste composition. The premixed barium cement paste may also contain a number of impurities from the original raw materials, preferably in an amount less than 10% of the paste in the cement composition or 30% of the cement powder in the cement composition. Such impurities may include, for example, iron oxides, magnesia (MgO), potassium oxide, sodium oxide, sulfur oxides, carbon dioxide, water, etc.

As noted above, the injectable premixed cement paste of the present invention does not set and harden in a hermetically sealed package because the reactions between hydratable barium cement compound and water only take place when exposed to an aqueous environment. After the cement paste is placed in contact with a physiological solution, a diffusional exchange of the non-aqueous carrier with the aqueous physiological solution, thereby exposing the premixed paste water and initiating the chemical reaction that transitions the cement to a hydrogel.

According to various embodiments, the premixed barium cement paste can be prepared by physical mixing processes (nonreactive), chemical mixing processes (reactive), biological mixing processes, and combinations thereof. For example, a premixed cement paste can be prepared by mixing the solid phases and water-free liquid using a ball mill process. The coupling agents are deposited on the solid powder surfaces by physical and chemical absorption, improving stability of the premixed paste. The coated solid particles are then mixed with the water-free liquid by ultrasound mixer to create a uniform paste. According to another embodiment, the flowability and injectability of the premixed barium cement paste are improved by controlling the particle size distribution of the solid components in the paste. In an embodiment, the particle size of the cement solids is in the range of between about 0.001 μm and about 100 μm. According to another embodiment, the particle size is in the range of between about 0.01 m and about 50 μm. According to an embodiment, organic dispersant agents (coupling agents) are introduced into the paste to improve the stability and injectability of the paste, including, for example, citric acid, sodium citrate, tartaric acid, succinic acid, phosphoric acid, dextrose, mannitol, sucrose, maltose, galactose, lactose, soluble starch, glucose, chitosan, galactose, amylopectin, celluloses, hydroxypropyl methyl cellulose, polyacrylic acids, carbonylmethyl cellulose, biopolymers, organic acids, silane, and mixtures of thereof. According to another embodiment, the organic dispersant agents comprise less than about 5% of the premixed paste.

According to an embodiment, antimicrobial agents are incorporated into the cement, including the premixed paste and powders. Any of a number of antimicrobial agents can be employed, including, for example, chlorine compounds, formaldehyde, glutaraldehyde, hydrogen peroxide, iodophors, peracetic acid, phenolics, chlorhexidine gluconate, lodine, quaternary ammonium compounds, nano-silver, or mixtures thereof. According to an embodiment, the antimicrobial agents comprise less than about 10% of the cement. Hereafter, a number of examples of tests performed by the inventors are described, exploring characteristics of various embodiments.

### EXAMPLES

### Example 1: Process for Making Barium Silicate Cement

The precursor material used to prepare the testing batches were 20 g tetraethyl orthosilicate (TEOS) (Sigma-Aldrich), 70 g barium acetate (Ba(C₂H₃O₂)₂), and 1 N nitric acid (Sigma-Aldrich). The chemical procedure for barium-silicate precursor was the barium acetate dissolved into 100 ml water and TEOS which was slowly added into barium acetate water solution. 2 g 1N nitric acid was then added as a catalyst of hydrolysis of the TEOS. The gelation of TEOS occurred rapidly (within a few hours). Neither precipitation nor phase separation occurred during the sol-gel transition. Finally, gels were dried in a furnace at 110° C. for about 24 hrs., then the mixture was calcined at 550° C. for 4 hrs., and finally the powder was placed in a platinum crucible for firing, at 1400° C. for 2 hrs. The barium silicate cement power comprised a mixture of 80% di-barium silicate and 20% tricalcium silicate. The cement powders were used for physical, chemical, and biological evaluations, including those described in the examples below.
Bench testing results:
Setting time: 2 hrs.
Compressive strength in 24 hrs.: 60 MPa

### Example 2: The Bioactivity of barium silicate Cement

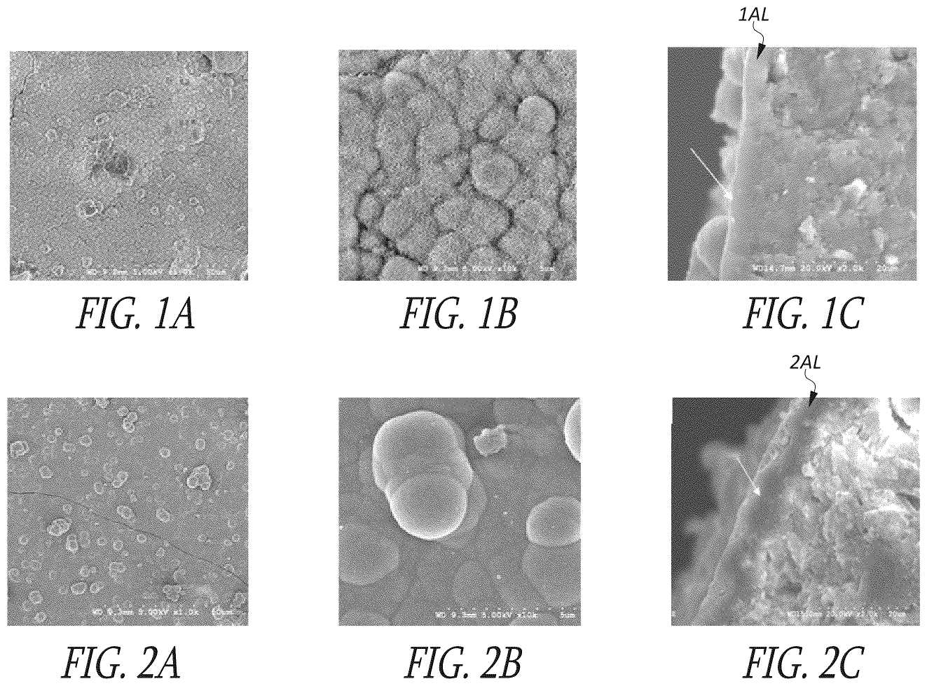

For in vitro bioactivity of barium silicate cement, a sample was prepared by mixing 1 g barium silicate cement powder with 0.6 g distilled water. A tricaclium silicate cement sample was used as a positive control sample. 7-day set sample discs (10 mm in diameter by 2 mm thick) were immersed in simulated body fluid (SBF) at 37° C. for 7 days. The ratio of SBF volume (ml) to the sample surface area (mm²) was approximately 0.1. Half the volume of the SBF solution was renewed every 24 hrs. during the soaking period. After being soaked for 7 days, the samples were characterized using XRD and SEM equipped with energy dispersive X-ray spectroscopy analyser (SEM/EDX; Hitachi S-3000N, Electronic System Ltd, Tokyo, Japan). SEM results clearly indicated that a 2 m thick layer of calcium hydroxyapatite was formed on the surface of both the barium silicate cement samples and the tricalcium silicate control sample as show in FIGS. 1A-C and 2A-C.

FIG. 1A is a scanning electron microscope (SEM) micrograph image showing a surface plan view of a barium silicate cement. FIG. 1B is a SEM micrograph image showing the surface morphology of a sample of barium silicate cement hydrogel after immersion in simulated body fluid (SBF) for 7 days. FIG. 1C is a SEM micrograph image showing a cross-sectional view of the barium silicate sample of FIG. 1B, showing a hydroxyapatite layer 1H that formed on the barium silicate sample during the immersion, a thickness of which was measured to be approximately 2 μm.

FIGS. 2A-2C are substantially similar to FIGS. 1A-1C, respectively, except that they show images of a tricalcium cement hydrogel control sample, in corresponding views. In FIG. 2C, a hydroxyapatite layer 2H was also formed, also approximately 2 μm in thickness.

It is well known in the art that hydroxyapatite can anchor and encourage new bone growth in appropriate physiological environments. The apatite formation on the test samples in SBF is therefore a strong indicator of the potential for in vivo bioactivity of the barium silicate and tricalcium silicate cement hydrogels.

### Example 3: Cytotoxicity of barium silicate Cement

Both cytotoxicity and cell adhesion were evaluated with human gingival fibroblasts (seventh to eighth passage), due to the availability of the cells. The cells were obtained from previously established stocks and cultured from healthy patients. (Dulbecco's Modified Eagle's Medium (DMEM; Gibco, Grand Island, NY), containing 100 μg/mL penicillin G, 50 μg/mL streptomycin, 0.25 ug/mL fungizone, and 10% fetal bovine serum (Gibco, Grand Island, NY), was used as the cell culture medium. Considering the resemblance to clinical applications for injectable cements, which set in the body, the cytotoxicity of tricalcium silicate samples and barium silicate cements was evaluated using an extract of freshly mixed cement paste. After mixing at w/c=0.5 ml/g, the cement pastes were immediately placed into 24-well tissue culture plates (Sarstedt Inc, Newton, NC) at 300 mg/well and exposed to ultraviolet light for 20 min before 1.5 ml of DMEM was added per well. The extracts were obtained and filtered (0.22 μm) after extraction for 24 hrs. and 3 days at 37° C. in 100% RH atmosphere containing 5% CO₂, and then, to observe a possible dose-dependant effect, the extracts were serially two-fold diluted using DMEM, and each dilution had five parallel experiment groups and one background group. The viabilities of human gingival fibroblast cells after exposure to 24 hr. and 3 day extracts of freshly mixed tricalcium silicate and barium silicate pastes for 3 days was assessed. Cell viability was strongly affected by cement composition (p<0.001 for all factors), but all materials behaved in a similar way. The biocompatibility of barium silicate cement was found to be equivalent to tricalcium silicate cement in terms of cell adhesion, proliferation, and cell cytotoxicity.

### Example 4: Strontium silicate and barium silicate Cement System

Strontium silicate was made by mixing 50 g strontium nitrate and 12 g Silicon dioxide nano-powder, and then the mixture was fired at 1550° C. for two hrs. The powder was ground in a ball mill with 99.9% ethanol alcohol for two hrs.

The slurry was dried at 110° C. for 24 hrs. barium silicate powder was made by following the process of example 1. Strontium silicate and barium silicate cement was made by mixing 20 g barium silicate, 10 g strontium silicate, and 1 g sodium silicate (accelerator) with a ball mill. The setting time of strontium silicate and barium silicate cement is 60 min, and compressive strength is 80 MPa.

### Example 5: Calcium Silicate, Calcium Phosphate, and Barium Silicate Cement System (CCB Cement)

The raw material of the calcium silicate was premixed by a sol gel process. 500 g calcium nitrate (Sigma-Aldrich) was dissolved in 2000 ml ethanol, and then adding 300 g TEOS (Sigma-Aldrich) and 36 g water, and the solution was continuously stirred for 48 hrs. The gelation of the solution took around 3-4 days. The Ca—Si gel was dried at 110° C. for 24 hrs., fired at 550° C. in alumina crucible for 4 hrs., and then fired at 1400° C. in zirconia crucible for 2 hrs. The calcium silicate powder contained 66% tricalcium silicate and 34% dicalcium silicate. The CCB cement was premixed by mixing 20 g barium silicate (prepared as in example 1), 8 g calcium silicate, 2 g di-calcium phosphate, and 0.5 g sodium silicate in a ball mill for 1 hr. The CCB cement was found to have excellent biocompatibility and bioactivity, high mechanical strength (150 MPa), short setting time (30 min.), and high radiopacity (equivalent to a 7.4 mm thickness of aluminum).

### Example 6: Premixed Paste of Barium Silicate

The barium silicate (60% tri-barium silicate and 40% di-barium silicate) was prepared by following the process in example 1. The premixed paste was prepared by mixing 50 g barium silicate powder with 14 g PEG 300 in ball mill for 20 min. The paste was loaded into a 1.0 ml syringe for clinical application. It was determined that the barium silicate cement paste was injectable, white colored, and suitable for dental applications, such as root canal filling, root-end filling material, retrofilling materials, pulp capping, apexification, and the sealing of perforations. The premixed barium silicate cement paste was evaluated according to ISO standard 6867:2012:
Setting Time: around 3 hrs.
Working time: >30 min.
Radiopacity: Equivalent to 9.0 mm of Al
Flowability: 25 mm
Film thickness: 50 μm
Solubility: <2.0%
Dimension Change: 0.01%

### Example 7: Preparation of Phosphate-containing Barium Silicate Paste

In this example the calcium phosphate and barium silicate paste were prepared synthetically using well defined pure chemicals. The raw materials used for the preparation of calcium phosphate-barium silicate powder were colloidal silica (50% Ludox®, 3M) for SiO₂, barium nitrate (99.9%, Sigma-Aldrich) for BaO, tetracalcium phosphate (Ca₄(PO₄)₂O), and dicalcium phosphate anhydrate (CaHPO₄H₂O) (Fisher®). Alternatively, and with no effect on the final properties of paste, the colloidal silica can be derived from thermal decomposition of hydrated silicon alkoxide such as TEOS, or added as fine pure silica powder. The designed composition of the cement was 65% tri-barium silicate, 20% di-barium silicate, 10% tetracalcium phosphate, and 5% dicalcium phosphate. A 200 g batch was prepared by mixing 96.32 g of colloidal silica, 160.98 g of barium nitrate and 1000 g distilled water in an alumina jar, and ball milled for 24 hours. The slurry mixture was dried using a spray dryer, and was then fired in a high temperature furnace at 1550° C. for 1 hour to form a mix of tribarium silicate and dibarium silicate The mixture was naturally cooled to room temperature. The resulting cement clinker was ground to −325 sieve particle size (<45 μm particle size), with average particle size of about 10 μ. 11.25 g of dicalcium phosphate anhydrate was dried in a furnace at 140° C. for 24 hours and then mixed with 20 g of the tetracalcium phosphate and with the fired cement powder (168 grams) in an alcohol solution by ball milling for 24 hours. The resulting slurry was spray dried. The average particle size of the cement powder was about 10 μm. The paste was prepared by mixing 200 g calcium phosphate and barium silicate powder, 30 g polyethylene glycol (molecular weight 400, Sigma-Aldrich), and 0.5 g hydroxypropyl methyl cellulose, in a planetary ball for 10 minutes. The hydroxypropyl methyl cellulose is a gelling agent for improving the viscosity and flowability of the phosphate-containing paste. The setting time of the paste at 37° C. in 100% humidity environment was about 10 hours. The average compressive strength after 7-day setting at 37° C. and 100% humidity was 101 MPa, with the standard deviation of 8 MPa. The cement paste was injectable and suitable for dental and orthopedic applications.

### Example 8: Preparation of Calcium phosphate—Calcium Silicate-barium silicate—Zirconia Paste

The raw materials were obtained and processed as described in previous examples. The powder mixture comprised 10% calcium phosphate, 40% Zirconia, 24% calcium silicate, 20% barium silicate, 5% sodium silicate, and 1% hydroxypropyl methyl cellulose. The paste was made by mixing 100 g powder and 26 g dimethyl sulfate in an ultrasonic mixier for 10 min. The paste was loaded into a 1 ml syringe.

The premixed barium silicate cement paste was evaluated according to ISO standard 6867:2012:
Setting Time: around 20 min
Working time: >30 min
Radiopacity: Equivalent to 8.5 mm of Al
Flowability: 25 mm
Film thickness: 50 μm
Solubility: <2.0%
Dimension Change: 0.01%

Where the claims recite the term medical, or related terms, this includes within its scope dental, unless the scope is explicitly defined otherwise.

The abstract of the present disclosure is provided as a brief outline of some of the principles of the invention according to one embodiment, and is not intended as a complete or definitive description of any embodiment thereof, nor should it be relied upon to define terms used in the specification or claims. The abstract does not limit the scope of the claims.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification, but should be construed to include all possible embodiments along with the full scope of equivalents to which such claims are entitled. Accordingly, the claims are not limited by the disclosure.

1. A material for use in medical applications, comprising:
a premixed paste, including:
a primary cement compound that includes one or more barium cements that are capable of a hydration reaction with water to form a hydrogel;
a nonaqueous liquid carrier that is hydrophilic and able to undergo an exchange with a water-based liquid when the premixed paste is exposed to an environment where water-based liquids are present.

2. The material of claim 1, wherein the premixed paste is packaged in a hermetically-sealed container.

3. The material of claim 1, wherein the one or more barium cements includes one or more of di-barium silicate (Ba₂SiO₄), tri-barium silicate (Ba₃SiO₅), mono-barium aluminate, di-barium aluminate, and tri-barium aluminate.

4. The material of claim 1, wherein the one or more barium cements comprise at least 2% of the barium cement, by weight.

5. The material of claim 1, wherein the one or more barium cements comprise at least 20% of the barium cement, by weight.

6. The material of claim 1, wherein the one or more barium cements comprise at least 50% of the barium cement, by weight.

7. The material of claim 1, wherein the one or more barium cements comprise at least 65% of the barium cement, by weight.

8. The material of claim 1, wherein the one or more barium cements comprise at least 80% of the barium cement, by weight.

9. The material of claim 1, wherein the premixed paste includes a secondary cement compound that includes one or more of calcium silicate compounds, calcium aluminate compounds, barium aluminate compounds, calcium phosphate compounds, strontium silicate compounds, strontium aluminate compounds, alkali silicate compounds, alkali aluminate compounds, magnesium silicate compounds, strontium aluminate compounds, lithium silicate compounds, sodium silicates compounds, potassium silicate compounds, and/or ruthenium silicate compounds.

10. The material of claim 9, wherein the one or more secondary cement compounds comprise no more than 50% of the barium cement, by weight.

11. The material of claim 1, wherein the nonaqueous liquid carrier comprises no more than 50% of the premixed paste, by weight.

12. The material of claim 1, wherein the nonaqueous liquid carrier comprises one or more of ethyl alcohol, ethylene glycol, polyethylene glycol, glycerol liquid, glycerin, liquid organic acids, water-soluble vegetable oil, water-soluble animal oil, fish oil, water-soluble silicon oil, dimethyl sulfoxide, and propylene glycol.

13. The material of claim 1, wherein the premixed paste includes one or more additional secondary, compounds.

14. The material of claim 13, wherein the one or more additional secondary, compounds include one or more of ceramics, ceramic fibers, polymers and polymer fibers, metals, metal salts, metal oxides, hydroxide compounds, non-oxide ceramics, biopolymers.

15. The material of claim 1, wherein the premixed paste includes an organic dispersant agent.

16. The material of claim 15, wherein the organic dispersant agent includes at least one of citric acid, sodium citrate, tartaric acid, succinic acid, phosphoric acid, dextrose, mannitol, sucrose, maltose, galactose, lactose, soluble starch, glucose, chitosan, galactose, amylopectin, celluloses, hydroxypropyl methyl cellulose, polyacrylic acids, carbonylmethyl cellulose, biopolymers, organic acids, and silane.

17. A method of using a cement material in medical applications, comprising:
preparing a cement, which includes a barium cement powder, for use in a dental procedure;
performing the dental procedure, including introducing the cement into a patient during the performance of the procedure; and
reacting the cement with an aqueous solution to form a hydrogel.

18. The method of claim 17, wherein the preparing a cement, which includes a barium cement powder, for use in a dental procedure comprises preparing the cement, which includes a barium cement powder comprising one or more of a di-barium silicate (Ba₂SiO₄), a tri-barium silicate (Ba₃SiO₅), a mono-barium aluminate, a di-barium aluminate, and a tri-barium aluminate.

19. The method of claim 17, wherein the reacting the cement with an aqueous solution comprises binging the cement into contact with the aqueous solution.

20. The method of claim 19, wherein the binging the cement into contact with the aqueous solution comprises mixing the cement with the aqueous solution prior to introducing the cement into the patient.

21. The method of claim 19, wherein the binging the cement into contact with the aqueous solution comprises binging the cement into contact with biological fluids present within the patient.

22. The method of claim 17, wherein:
the preparing a cement, which includes a barium cement powder, for use in a dental procedure comprises removing from a package a premixed paste that includes the barium cement powder mixed with a nonaqueous liquid; and
the introducing the cement into a patient during the performance of the procedure comprises introducing the premixed paste into the patient.

23. The method of claim 22, wherein the reacting the cement with an aqueous solution comprises, after introducing the premixed paste into the patient, exchanging biological fluids of the patient with the nonaqueous fluid of the premixed paste.
